(19) United States
(12) Patent Application Publication
(10) Pub. No.: US 2008/0277080 A1
(43) Pub. Date: Nov. 13, 2008

(54) Method for the Genetic Modulation of the Biosynthesis of Hemicelluloses, Cellulose and Uronic Acids in Plant Cells Using Gene Expression Cassettes

(75) Inventors: Carlos Alberto Labate, Sao Paulo (BR); Monica Teresa Venesiano Labate, Sao Paulo (BR); Ana Leticia Bertolo, Sao Paulo (BR); Daniela Defavari Nascimento, Sao Paulo (BR); Gunta Gutmanis, Sao Paulo (BR); Maria Inez Fernandes Faraldo, Sao Paulo (BR)

Correspondence Address: MCDERMOTT WILL & EMERY LLP, 600 13TH STREET, N.W., WASHINGTON, DC, 20005-3096, US

(73) Assignees: Suzano Bahia Sul Papel E Celulose S.A., Pituba- Salvador, Bahia (BR); Fundacao De Estudos Agrarios"Luiz De Queiroz" - FealQ, Piracicaba-SP (BR); Fundacao De Amaparo A Pesquisa Do Estado De Sao Paulo, Sao Paulo-Sp (BR)

(21) Appl. No.: 10/586,875

(22) PCT Filed: Jan. 21, 2005
(86) PCT No.: PCT/BR2005/000008

§ 371 Date: Jun. 5, 2007

(30) Foreign Application Priority Data
Jan. 22, 2004 (BR) PI0400246-6

(51) Int. Cl. D21C 9/00; C07H 21/00; C12N 15/82; C12N 15/89; C08B 15/00; C12N 15/87; A01H 1/00; A01H 5/00

(52) U.S. Cl. 162/1; 536/23.2; 435/468; 435/470; 435/469; 800/278; 800/298; 536/56; 162/100

(57) ABSTRACT

The present invention refers to gene expression cassettes encoding enzymes involved in the metabolic pathway for the biosynthesis of hemicelluloses, cellulose and/or uronic acids, and to a method for genetic transformation of plant cells through the introduction of one or more gene expression cassettes from the present invention, and the overexpression and repression of these genes in plants.

More particularly, the present invention refers to a method for introducing expression cassettes in plants. Additionally, the present invention refers to genetically modified plants.

The present invention also refers to the attainment of the genetically modified plant, its derived plants and seeds, as well as the wood, paper and cellulose from this plant.

### FIELD OF THE INVENTION

The present invention refers to gene expression cassettes encoding enzymes involved in the metabolic pathway for biosynthesis of hemicelluloses, cellulose and/or uronic acids, and to a method for genetic transformation in plant cells through the introduction into the said cells of one or more gene expression cassettes. In addition, alteration in metabolite content and/or composition of this metabolic pathway in plants or parts of the plant.

The invention relates to isolated genes and the respective encoded enzymes involved in the biosynthesis of hemicelluloses, cellulose and/or uronic acids.

The present invention also refers to a method for obtaining genetically modified plant, its derived plants and seeds, as well as the wood, paper and cellulose derived from this plant, and their uses.

### BACKGROUND OF THE INVENTION

It is known that the chemical composition of cellulose pulp, primarily related to the presence of hemicelluloses, determines the physical properties of the paper, as well as the physico-chemical and optical properties (kappa, viscosity, yield, whiteness and whiteness reversion) during the process of cellulose production.

Cellulose is the most abundant polysaccharide in the cell wall of the plant kingdom. It is a linear polymer containing anhydrous glucose β(1→4) units. It is the main component of wood, and therefore of paper. Other plants also have a great amount of fibers in the cell wall, such as cotton, for example. The cellulose molecules are organized in bundles of parallel chains forming fibrils, which are linked to each other by the polymeric material hemicellulose and other substances.

Another important component of the cell wall is hemicellulose. Hemicelluloses are composed of several polysaccharides such as glucans, xylans, xyloglucans, and mannans. Such polysaccharides composed of polymers containing two or more of the following sugars: D-xylose, D-mannose, D-glucose, D-galactose, L-arabinose, D-glucuronic acid and its derivative 4-O-methylglucuronoxylan, and D-galacturonic acid (Vickery & Vickery, (1981) Secondary plant metabolism. pp. 335, Macmillan Press Ltd., London). This group of polysaccharides is characterized by a central structure formed by residues of 1,4 β-xylan, and by ramifications composed of glucuronic acid molecules, arabinose and manans.

It is known that the concentration of hemicelluloses in the cellulose pulp is mainly affected by the active alkali in the cooking of wood, so that when a greater concentration of this reagent is used, a lower retention of hemicelluloses is observed, as well as change in the physico-chemical properties (kappa, viscosity, cellulose yield, and whiteness).

Hemicelluloses can be classified according to the quantity of uronic acid in acid hemicellulose, which presents a large amount of this acid, and neutral hemicellulose, when it has a low concentration of uronic acid.

In addition, uronic anhydride concentrations affect the kappa number and the ISO whiteness of the obtained cellulose pulp, indicating the formation of complexes that give color to the pulp, and not only to the remaining lignin fragments.

Hemicellulose retention allows the production of pulps with differential physico-mechanical properties and fibers with different surface composition, thus influencing printing and packaging characteristics of the paper.

Hemicellulose biosynthesis, as well as that of the other cell wall components, depends on the supply of the sugars (UDP-glucose phosphorylated sugar) produced by photosynthesis in the chloroplasts. The carbon fixed by photosynthesis is exported from the chloroplast to the cytoplasm in the form of triose-phosphate (dihydroxyacetone phosphate or DHAP), starting a series of metabolic reactions in which the flow direction will depend on several factors, such as the leaf development stage, physiologic status of the plant and nutritional condition, among others. In young leaves, a great part of the metabolic flows is preferentially used for the synthesis of lipids, through pentose phosphates (for the synthesis of nucleic acids), glycolysis (generating ATP and pyruvate), tricarboxylic acid (TCA) cycle, amino acid synthesis, and the sugar oxidation pathway, responsible for the synthesis of the cell wall components.

Another important component in hemicellulose biosynthesis is the myo-inositol oxidation pathway (Loewus F. A. et al., (1962) “Metabolism of myo-inositol in plants: conversion to pectin, hemicellulose, D-xylose, and sugar acids. Proc Natl Acad Sci USA, 48: 421-425; Loewus F A and Dickinson D B (1982) Cyclitols. In: Encyclopedia of plant physiol., new series, Vol. 13A, Carbohydrates I: Intracellular Carbohydrates, F. A. Loewus, W. Tanner eds., p. 193-216; Loewus F. A. et al., (2000) “Myo-inositol metabolism in plants”. Plant Science, 150:1-19). Myo-inositol is formed from glucose-6-P, the same precursor of UDP-glucose. This compound is an important precursor for numerous metabolic products, such as inositol phosphates, phosphoinositides, methylated derivatives and IndoleAcetic Acid (IAA) conjugates, and glucuronic acid. The first step in the production of inositol involves the synthesis of myo-inositol-1-phosphate (Ins-1P) from D-glucose-6-P, catalyzed by the myo-inositol 3-phosphate synthase enzyme (MIPS, EC 5.5.1.4). The produced inositol-1-P is then dephosphorylated by the enzyme inositol monophosphatase (IMP, EC 3.1.3.25) to release myo-inositol. These two metabolic steps determine the formation of an essential compound for the normal growth of cells and other essential functions. The reduction in myo-inositol levels in Saccharomyces cerevisiae cells does not allow normal growth (Henry S. A. et al, (1977) “Growth and metabolism of inositol-starved Saccharomyces cerevisiae”. J. Bacteriol., 130: 472-484). In plant cell and growing tissue cultures, reduction in the intracellular amounts of myo-inositol inhibits cell division (Biffen, and Hanke, (1990) “Reduction in the level of intracellular myo-inositol in culture soybean (Glycine Max) cells inhibits cell division”. Biochem. J., 265: 809-814; Loewus and Dickinson, above; Loewus and Loewus (1983) “Myo-inositol: its biosynthesis and metabolism”. Annu. Rev. Plant Physiol., 34: 137-161; Biswas et al., (1984) “Myo-inositol polyphosphates and their role in cellular metabolism: a proposed involving glucose-6-phosphate and myo-inositol phosphates”. In: Subcellular Biochemistry (Roodyn, D. B., ed.). Plenum Press, London, 237-280; Loewus (1990) “Inositol biosynthesis”. In: D J Moore, W F Boss, and F. Loewus (ed.), “Inositol metabolism in plants”. Wiley-Liss, New York, p. 13-19). The reduction in myo-inositol biosynthesis was observed in genetically modified potato plants with suppressed MIPS enzyme activity, using an antisense expression of the cDNA StIPS that encodes the potato gene. Reduction in the activity of this enzyme in the leaves, about 20% of the levels observed in the wild plants, resulted in a strong reduction in inositol, galactinol and raffinose content (approximately 7%, 5%, and 12%, respectively). The genetically modified plants presented reduction in apical dominance, changes in morphology, early leaf senescence, and reduction in the weight of the tubercules. These results show the importance of myo-inositol metabolism in plant physiology and development.

It is known that, in order to change agronomic traits, generally polygenic, in nature, multiple genes are involved in trait expression. In the case of metabolic pathways, enzymes encoded by independent genes, acting together, convert substrates into a product. A way to change the metabolic pathway, according to the invention, is through the introduction of the said gene expression cassettes that are introduced into plant cells.

In the case of wood, the main raw material used in the production of cellulose pulp, significant differences are observed with regard to the level and the monomeric units that within the hemicelluloses, between trees from the hardwood and coniferous groups, and therefore, in the chemical composition of the resulting pulps (Rydholm, (1965) “Pulping Processes”, Interscience Publishers, New York, 3, p. 254). The unbleached pulp of eucalyptus, obtained in a kraft pulping process, is predominantly formed by a mixture of carbohydrates and a small fraction, about 3%, of lignin polymer fragments.

Studies performed with wood from coniferous trees, indicate that it is possible to promote hemicellulose retention during the pulping process (Genco et al., (1990) “Hemicellulose retention during kraft pulping”. TAPPI Journal, April: 223-233). The increase of the hemicellulose concentrations in pulps, according to Macintosh, D.C. (1963) TAPPI Journal 46(5): 273 and Spielberg, H. (1966) “The effect of hemicellulose on the mechanical properties of individual pulp fibers”. TAPPI Journal, 49(9): 388, affects the physico-mechanical properties that increase elasticity, force required for rupture and length before auto-rupture.

Foelkel, C. (1997) “Qualidade da madeira de eucalipto para atendimento das exigencias do mercado de celulose e papel” (“Quality of eucalyptus wood to meet the demands of the paper and cellulose market”). In: IUFRO Conference on Silviculture and Improvement of Eucalypts, Salvador, Anais. Colombo: EMBRAPA/CNPF, v. 3, p. 15-22, demonstrated that the processes involved in wood quality are directly related to changes in the production scale, making the forest and wood quality breeding programs, especially for eucalyptus, present quantitative aspects and facilitating procedures. The genetic improvement programs are common, principally in the forest area, generating trees that produce 12 to 16 tons of kraft cellulose per hectare per year. Significant yields are obtained through volumetric growth, wood density, lignin content and the easiness of delignification, and the yield of purified cellulose after cooking. However, breeding programs that go beyond the agronomic traits, such as developing wood with characteristics related to paper bleaching and final product features, are rare (Foelkel, C, above).

The energy consumed in pulp refining is also affected by hemicellulose concentration through the insertion of water in the crystalline areas of the fibers (Giertz, H. W. (1948) “Cellulosa och Papper”, SPC, 1908-1948 Review, Stockholm, 417: 1908-1948). This phenomenon decreases the energy consumed during refining, besides economic benefits, products with enhanced physico-mechanical properties are also obtained.

Hemicellulose concentration also affects the chemical composition of fiber surface. Isogai et al. (1997) “Effects of carboxyl groups in pulp on retention of alkylketene dimmer”. Journal of Pulp and Paper Science, 23(5): 215, indicate that the presence of carboxylic acids can affect the reactions with alkylketene dimmers, a compound used in paper gluing. The functional groups present on the fiber surfaces also affect fiber-fiber interactions (Carlsson, G. (1996) “Surface composition of wood pulp fibres-relevance to wettability, sorption and adhesion”. Dissertation Thesis, Royal Institute of Technology, Stockholm, Sweden), fiber-additives for coating (Biermann et al., (1997) “A new sizing agent: Styrene-maleic anhydride copolymer with alum mordants”. TAPPI Journal, 80:1-277), and the type of wood used affect the colloidal and polyelectrolyte properties (Rydholm, S. A., above), pulping conditions (Karlsson and Westermark, (1997) “The significance of glucomannan for the condensation of cellulose and lignin under kraft pulping conditions.” Nordic Pulp and Paper Research Journal, 12(2): 90p; Treimanis, A. (1996) “Wood pulp fiber structure and chemical composition, their influence on technological processes”. Nordic Pulp and Paper Research Journal, 3: 146p) and different reagents used in the bleaching stages (Laine and Stenius, (1996) “The effect of ECF and TCF bleaching on the surface chemical composition of kraft pulp as determined by ESCA”. Nordic Pulp and Paper Research Journal, 3: 201p).

The effects of hemicelluloses on the physico-mechanical and optical properties of the eucalyptus pulp were investigated by Milanez et al. ((1982) “Influência das hemiceluloses nas propriedades óticas e físico-mecanicas da polpa” (The influence of hemicellulose on the optical and physico-mechanical properties of the pulp). Proceedings of ABTCP, XV Congresso Anual, 155) who used the extraction method in 5% NaOH as an approximation for the hemicellulose content (pentosanes). The results obtained indicate that the increase in the concentration of the extractives at 5% NaOH causes a reduction in energy needed for refining and an increase in the physico-mechanical properties of the cellulose pulp.

It is known that eucalyptus kraft cellulose is utilized principally in the manufacture of two paper types: printing/writing and sanitary. Printing and writing papers should have properties such as brightness, opacity, formation, specific volume, porosity, printability, resistance, dimensional stability, among others (Foelkel, C., above).

The method chosen to insert exogenous genes into plant cells is important for the successful transformation of plants. The basis of these methods involves the insertion of the genes of interest into the host plant genome with a marker gene that allows for the selection of genetically modified cells, resistant to a certain antibiotic, herbicide or other selective agent, from non-genetically modified cells. These methods are usually efficient for the introduction of a gene into the host plant. Plant transformation in biotechnology programs consists of the introduction of a fragment (gene expression cassette) into the genome of the target cell. Fertile plants should be produced from a single transformed cell, and the inserted expression cassette should be capable of being transferred to the successive generations through the seeds.

### DESCRIPTION OF THE INVENTION

The present invention uses molecular biology techniques, tissue culture and gene transfer, to alter the expression of the genes, in both the sense and antisense orientation, coding for enzymes that participate in the metabolic pathway for the biosynthesis of hemicelluloses, cellulose and/or uronic acids. In addition, it refers to the isolation of genes responsible for the trait of interest, located and isolated from genes of other genomes, and introduced into binary vectors, for use in target plant transformation. Therefore, gene expression cassettes are introduced into plant cells, in a controlled manner, modifying the genome of the receptor organism, independent of the fecundation process.

The present invention, in one of its embodiments, refers to gene expression cassettes encoding enzymes related to hemicelluloses, cellulose and/or uronic acids biosynthesis, and the use of these cassettes.

The present invention also relates to the change in the expression of genes encoding the biosynthetic enzymes cited above, as well as to changes in the composition and/or content of hemicellulose, cellulose and/or uronic acids. It also refers to the modulation of the expression of genes encoding the enzymes involved in the biosynthesis of the compounds cited above, as well as to their concentration and/or these polypeptide levels in these plants or parts of these plants. The modulation is affected by the increase or decrease in the concentration and/or level of these polypeptides, of the present invention, in plants.

The present invention also refers to a method of genetic transformation in plant cells in which one or more gene expression cassettes encoding enzymes related to the biosynthesis of hemicelluloses, cellulose and/or uronic acids are introduced.

The present invention also refers to genetically modified plants and to the method for obtaining these plants. It also concerns plants derived from the genetically modified plants, that have one or more gene expression cassettes encoding enzymes related to the biosynthesis of hemicelluloses, cellulose and/or uronic acids.

The present invention also refers to genetically modified seeds from the genetically modified plant and, finally, it refers to the wood and cellulose of from the said genetically modified plant, and their uses.

The method for plant cell genetic transformation of the present invention use well-known techniques such as bioballistics, electroporation, microinjection, macroinjection or Agrobacterium tumefaciens-mediated transformation, for the introduction of the interest gene expression cassettes, for both the repression, as well as the overexpression of the said genes in the target cell. Preferably, but not limited to just this embodiment, the cassette is cloned into the binary vector and used to genetically transform Agrobacterium tumefaciens. This bacterium is naturally found in the soil, and it is considered as natural engineer, being an efficient vector for the transformation of plants.

According to the invention, gene expression cassettes are introduced into plant cells, in a controlled manner, modifying the genome of the receptor organism, independent of the fecundation process. The gene responsible for the trait of interest is located and isolated from other genes of the genome, and introduced into binary vectors for target cell transformation. This totipotent phenomenon allows genetically modified plants to be obtained from the originally transformed cells with chimerical constructions.

The said gene expression cassettes, according to the present invention, carrying out the overexpression, by sense orientation, or repression, by antisense orientation, of one or more genes, encoding the said enzymes of interest.

The genetically modified plant, according to the present invention, contains one or more of the gene expression cassettes encoding the said enzymes of interest, the genes being expressed in either the sense or antisense orientation.

The present invention also presents a method for obtaining the genetically modified plant, which comprises the stages of:

(a) genetic transformation of plant cells through the introduction of genes expression cassettes encoding the enzymes of interest of the present invention, by bioballistics, electroporation, microinjection, macroinjection or Agrobacterium tumefaciens-mediated transformation, for instance;

(b) regeneration of stage (a) cells;

(c) expression of the DNA constructed in the cells of stage (b) in a sufficient amount to substantially change the metabolic pathway of the hemicelluloses and/or cellulose and/or uronic acids biosynthesis; and

(d) obtention of the modified plant.

The introduction of one or more gene expression cassettes in the plant cells causes alterations in the metabolic pathway of hemicelluloses, particularly xylans, cellulose and/or uronic acids, particularly glucuronic acid, altering the yielding of the process, chemical consumptions and product quality, according to the industrial interest. Thus, the present invention improves the wood quality for cellulose and paper production applications.

An object of the present invention is to improve the wood quality by altering the chemical composition of the plant cell wall fibers, with focus on angiosperms, particularly, eucalyptus, and gymnosperms, by altering the metabolic pathway of hemicelluloses, cellulose and/or uronic acids. More specifically, the metabolic pathway change is obtained by altering the expression of one or more genes, separately or together, in both sense and antisense orientation, responsible for the enzymes that participate in the hemicelluloses, cellulose and/or uronic acids biosynthesis. This is obtained by introducing gene expression cassettes encoding enzymes capable of altering the metabolic pathway of hemicelluloses, cellulose and/or uronic acid, particularly by increasing the proportion of xylans and/or reducing and/or increasing the uronic acid content.

The enzymes of interest involved in the hemicelluloses, cellulose and/or uronic acids biosynthesis, according to the present invention, are selected from the group that contains: myo-inositol 1-phosphate synthase (EC: 5.5.1.4), myo-inositol monophosphatase (EC: 3.1.3.25), myo-inositol oxygenase (EC: 1.13.99.1), β-glucuronidase (EC: 3.2.1.31), glucuronokinase (EC: 2.7.1.43), glucuronosyltransferase (EC: 2.4.1.17), glucuronate-1-phosphate uridyltransferase (EC: 2.7.7.44), phosphoglucomutase (EC: 5.4.2.2), UDP-glucose pyrophosphorylase (EC: 2.7.7.9), UDP-glucose dehydrogenase (EC: 1.1.1.22), UDP-D-glucuronate carboxylase (EC: 4.1.1.35), 1,4-β-D-xylan synthase (EC: 2.4.2.24), and cellulose synthase (EC: 2.4.1.1).

The reference “E.C.” followed by a number, in the above paragraph, refers to the IUBMB (International Union of Biochemistry Molecular and Biology) nomenclature of enzymes.

More specifically, the enzymes of interest according to the present invention are selected from the group containing: myo-inositol oxygenase (EC: 1.13.99.1), β-glucuronidase (EC: 3.2.1.31), glucuronokinase (EC: 2.7.1.43), glucuronate-1-phosphate uridyltransferase (EC: 2.7.7.44), UDP-glucose pyrophosphorylase (EC: 2.7.7.9), UDP-glucose dehydrogenase (EC: 1.1.1.22), and UDP-D-glucuronate carboxylase (EC: 4.1.1.35).

Another object of the present invention is wood with chemical qualities and physical properties distinct from the state of the art, obtained from the genetically modified plant according to the present invention, that allows a significant decrease in industrial process costs and improvements in the final product, such as paper, also finding use in civil engineering, shipbuilding, furniture manufacturing, appliances, as well as the cellulose pulp production, particularly used for the cellulose pulp and paper productions.

The present invention also directed towards other plants, within angiosperms and gymnosperms, apart from eucalyptus, such as wheat, for example, whole-wheat flour, extracted from wheat, presents an abundant amount of fibers on the cell wall. One can also mention cotton, to which changes can also be observed, since cotton is almost exclusively composed of cellulose. The sense or antisense cDNA expression is directly related to the metabolic activity of the main enzymes of hemicellulose biosynthesis and to the change in cell wall composition.

In the particular case of trees, hemicellulose retention allows the obtention of pulps with differential physico-mechanical properties and fibers with distinct surface composition, that influence characteristics of paper for printing and packaging and absorbing papers. Besides affecting the product characteristics, hemicellulose retention favors the performance of papermaking machines.

Another object of the present invention is cellulose obtained from the wood from the genetically modified plant and the use of the said cellulose for paper manufacturing that could have a use in civil engineering, furniture industry, printing paper, packaging, and absorbing paper, among others. In preference, the paper from the modified cellulose of the present invention finds uses as printing, packaging, and absorbing papers.

Furthermore, another object of the present invention is the genetically modified seeds that present one or more gene expression cassettes encoding the enzymes of interest of the present invention, and the use of these seeds to obtain genetically modified plants.

Another object of the present invention is to increase the yield of cellulose in the production process, as well as change the unbleached and bleached cellulose paste quality, modulating the uronic acids and/or cellulose and/or hemicelluloses content of the modified plants.

The use of transgenic methods is associated with conventional plant improvement programs, accelerating the process of obtaining products with specific and desirable characteristics for the industrial sector, which are related to forest improvement and plant genetic improvement.

The enzyme myo-inositol oxygenase has been recently cloned from a pig kidney cDNA library (Arner et al., 2001. “Myo-Inositol oxygenase: molecular cloning and expression of a unique enzyme that oxidizes myo-inositol and D-chiro-inositol”. Biochem. Journal, 360(2): 313-320). This sequence was used as basis for the synthesis of primers used in the amplification of orthologous regions present in the genome of the eucalyptus, thus facilitating the location of the miox gene in the tree genome. The enzyme of interest, β-glucuronidase was cloned from Escherichia coli and was expressed in both sense and antisense orientation, in Eucalyptus grandis.

According to the present invention, the term “genetically modified plant” obtained by the method cited above includes cell, organ, tissue, seed, the entire plant, or its progenies.

“Derived plants”, according to the present invention, refers to the plants derived from the genetically modified plants with one or more gene expression cassettes encoding the enzymes of interest of the present invention. The said derived plants present alteration in the chemical composition of the cell wall through the alteration in the metabolic pathway for the biosynthesis of hemicelluloses, cellulose and/or uronic acids.

The terms “sense expression” and “antisense expression”, according to the present invention refer to the overexpression or repression of the genes encoding the enzymes of interest, respectively.

According to the present invention, “gene expression cassette” refers to expression cassettes that, regardless of their construction, comprise genes encoding the enzymes of interest of the present invention.

The present invention proposes to change the biosynthesis of hemicelluloses, cellulose and/or uronic acids, thus improving the wood quality. In order to understand the strategies that have been used in this invention, it is necessary to know the biochemistry of hemicellulose biosynthesis and the enzymes that participates in the process.

The uronic anhydrides are beneficial in wood cooking, by protecting hemicellulose terminator groups, preventing peeling reactions. The correlation between uronic anhydrides and optical properties indicates that these components form chromophoric compounds that give a brown color to the pulp after conventional bleaching and thus consume more oxidizing reagents in the bleaching process. From these results, the applicant has developed the technology of the present invention, seeking to obtain plants, more particularly trees, with altered concentrations of xylans and uronic anhydrides.

### BRIEF DESCRIPTION OF THE FIGURES AND THE TERMS USED

The present invention is better understood from the following description of some terms and FIGURE that are part of the present invention.

a) E-Value: “Expected Value”—discriminates the background that occurs at random, that is present in the alignment between nucleotide sequences. The lower the E-value, or closer to zero, more significant is the alignment between the sequences. However, for short sequences, as may observe in the case of primer sequence analysis, high E-values may occur for virtually identical sequences. In this case, E-value estimation takes into account the length of the sequence in question, because a short sequence has a high probability of occurring purely by chance in the database.

b) Gi number: Access number of the gene in the NCBI (National Center for Biotechnology Information) database.

c) ORF: “Open Reading Frame” corresponds to the c-DNA.

d) Bit Score: Version scale of the alignment used in the statistical analysis of NCBI's BLAST system.

e) Cluster: Sequences organized in groups by homology.

f) EST: “Expressed Sequence Tag”—a short cDNA sequence (complement DNA).

g) FASTA: unformatted text, but with line breaks, containing 60 characters per line. Standard model for algorithmic comparison of a heuristic sequence, used for searching sequences in the database via sequence alignment.

h) FRAME NUMBER: possible reading frames (three in each sense: +1, +2, +3, −1, −2, and −3), found in a DNA fragment with the size of a gene, starting with the initiation codon ATG, and finishing with a terminator codon (TAA, TAG, or TGA).

i) nptII: nopaline phosphotransferase II gene, which confers resistance to kanamycin (Bevan et al., 1983);

j) htp: hygromycin B phosphotransferase gene that confers resistance to hygromycin (van den Elzen et al., 1985);

k) bar: phosphinothricin (bialaphos) acetyltransferase gene that confers resistance to ammonium glufosinate (Thompson et al., 1987);

l) BLAST—“Basic Local Alignment Search Tool”: Altschul et al., (1997) “Gapped BLAST and PSI-BLAST: a new generation of protein database search programs”. Nucleic Acids Research, 25: 3389-3402;

m) BLAST N: compares the nucleotide sequence to be analyzed against the available nucleotide sequences from the NCBI database.

n) BLAST X: compares the translated nucleotide sequence to be analyzed against the available protein sequences from the NCBI database.

o) BLAST 2 Sequences: performs the comparative analysis of the multiple alignments of 2 or more nucleotide or protein sequences.

FIG. 1 shows the several metabolic pathways in order to get cellulose, hemicelluloses and glucuronic acid. The key metabolite of the entire process is UDP-glucose that serves as raw material for the sucrose, cellulose and hemicellulose synthesis, thus being an important component in carbon partition among the several compounds.

FIG. 1 also shows the name of the enzymes, with their international numbering codes (E.C.), encoded by the genes that have been isolated in the invention, among other: EC: 5.5.1.4, myo-inositol 1-phosphatesynthase (MIPS); EC: 3.1.3.25, myo-inositol monophosphatase (IMP); EC: 1.13.99.1, myo-inositol oxygenase; EC: 3.2.1.31, β-glucuronidase; EC: 2.7.1.43, glucuronokinase; EC: 2.4.1.17, glucuronosyltransferase; EC: 2.7.7.44, glucuronate-1-phosphate uridyltransferase; EC: 5.4.2.2, phosphoglucomutase; EC: 2.7.7.9, UDP-glucose pyrophosphorylase; EC: 1.1.1.22, UDP-glucose dehydrogenase, EC: 4.1.1.35, UDP-D-glucuronate carboxylase; EC: 2.4.2.24, 1,4-β-D-xylan synthase, EC: 2.4.1.1, cellulose synthase.

A schematic diagram of the formation of xylans and glucuronic acids from glucose-6-P sugar, and of the oxygenation pathway of the enzymes cited in the previous paragraph is shown. The blank frames indicate the enzymes whose genes have not yet been cloned and sequenced from any organism.

### Method for Gene Expression Cassette Introduction in Plants

The description below is given merely for illustrative and exemplifying purposes, not being limitative, a way to isolate some of the genes of the invention, regardless of any changes that these may have, involved in the hemicelluloses, cellulose, and/or uronic acids biosynthesis:

1) UDP-glucose dehydrogenase gene (EC: 1.1.1.22);

2) uxs1 gene of the enzyme UDP-D-glucuronate carboxylase (EC: 4.1.1.35);

3) miox gene of the enzyme myo-inositol oxygenase (EC: 1.13.99.1);

4) gene of the enzyme UDP-glucose pyrophosphorylase (EC: 2.7.7.9);

5) gene of the enzyme glucuronokinase (EC: 2.7.1.43);

6) gene of the enzyme glucuronate-1-phosphate uridyltransferase (EC: 2.7.7.44);

A search was done using the EC number (enzyme code) in the NCBI (National Center for Biotechnology Information) database, at the website www.ncbi.nlm.nih.gov, which gathers the nucleotide sequences of already sequenced organisms, available for research. Links for analysis are provided, such as:

A) BLAST N: compares the nucleotide sequence to be analyzed against the available nucleotide sequences from the NCBI database.

B) BLAST X: compares the translated nucleotide sequence to be analyzed against the available protein sequences from the NCBI database.

C) BLAST 2 Sequences: performs the comparative analysis of the multiple alignment of 2 or more nucleotide or protein sequences.

The results allow to obtain the sequences of the encoding genes of the enzymes cited above, as follows:

1) EC: 1.1.1.22: gene of the enzyme UDP-glucose dehydrogenase from Glycine max (soybean) (Gene Bank accession number: U53418).

2) EC: 4.1.1.35: uxs1 gene of the enzyme UDP-D-glucuronate carboxylase from Pisun sativum (pea) (Gene Bank accession number: BAB409674).

3) EC: 1.13.99.1: miox gene of the enzyme myo-inositol oxygenase, from Sus scrofa (Gene Bank accession number: AAL39076).

4) EC: 2.7.7.9: gene of the enzyme UDP-glucose pyrophosphorylase, from Solanum tuberosum (Gene Bank accession number: U20345).

5) EC: 2.7.1.43: gene encoding the enzyme glucuronokinase.

6) EC: 2.7.7.44: gene encoding the enzyme glucuronate-1-phosphate uridyltransferase.

The sequence in question can be compared to the protein database using BLASTP. Within this option, comparison of conserved protein domains is offered using the CDS (“Conserved Domain Search”) against the CDD (“Conserved Domain Database”) database that contains the protein families.

For all the enzymes related above, gene sequences were found in the genomes of live organisms, with a high degree of protein homology and conservation of the domains indicated by CDS.

After identification of candidate sequence for each gene, these sequences were used for the synthesis of specific primers for each ORF with the help of the Primer 3 program (as the one found in the websites www.genome.wi.mit.edu/genome_software/other/primer3.html, or www.broad.mit.edu/cgi-bin/primer/primer3.cgi, for example), which allows the design of specific primers for a certain nucleotide sequence. These primers were used as initiators in the amplification of the entire coding region of the related genes, using the PCR (Polymerase Chain Reaction) technique. The product resulting from PCR reaction was sequenced and submitted to identity confirmation, against the NCBI databases, with the help of the BLAST Program. This PCR product was used as a probe in the Southern Blot technique to verify the presence of this sequence in the plants genome.

The isolation of the genes of interest, independent of any changes that these may have, was conducted using an ORF (Open Reading Frame) search, corresponding the enzymes cited above, for their subsequent cloning into binary vectors, with focus on the vector named Gateway, from the American company Invitrogen Corporation, but not limited to this vector. Primers were synthesized and the each enzyme corresponding ORF was predicted from the nucleotide sequences of these genes: Glycine max for UDP-glucose dehydrogenase, Pisum sativum for UDP-D-glucuronate carboxylase, Solanum tuberosum for UDP-glucose pyrophosphorylase, and Sus scrofa for myo-inositol oxygenase, using the NCBI BLAST Program.

The expression cassette containing the gene of interest was cloned into a binary vector for plant transformation, for overexpression or repression of this gene in plants, with focus on tobacco and eucalyptus, using the Agrobacterium-mediated plant transformation technique.

The gene sequences of the enzymes UDP-glucose dehydrogenase (EC 1.1.1.22), UDP-D-glucuronate carboxylase (EC 4.1.1.35), myo-inositol oxygenase (EC 1.13.99.1), UDP-glucose pyrophosphorylase (EC 2.7.7.9), glucuronokinase (EC 2.7.1.43), and glucuronate-1-phosphate (EC 2.7.7.44) were used for the synthesis of specific primers, encompassing the complete sequence of these genes. The reaction product was sequenced and submitted to identity confirmation with the help of the NCBI BLAST program. The cDNAs of the said enzymes, obtained as a RT-PCR product, using the Invitrogen “SuperScript™ Plasmid System with Gateway® Technology for cDNA Synthesis and Cloning” kit, according to manufacturer's recommendations, were cloned into Gateway vectors (Karimi et al., (2002) “Gateway™ vectors for Agrobacterium-mediated plant transformation.” Trends Plant Science, 7(5): 193-195), among others.

As in the cloning method using Gateway® Technology the PCR product is flanked by att recombination sites, it could be directionally cloned in both the sense or antisense orientation into this vector, which contains compatible recombination sites, in a reaction mediated by the Gateway® LR Clonase™ by Invitrogen.

The Gateway binary vector already has the markers genes nptII, htp, or bar, for transformant selection. All these selection genes are under transcriptional control of the nos promoter and terminator, nopaline syntethase (Hellens et al., (2000) “pGreen: a versatile and flexible binary Ti vector for Agrobacterium-mediated plant transformation.” Plant Molecular Biology, 42: 819-832), located on the left border of the T-DNA. The recombination site for the interest fragment insertion is located on the right border of the T-DNA.

In the specific case of cloning into the Gateway vector, the overexpression or repression of the cDNA sequences of the enzymes was done using the cloning site located on the T-DNA right border, between the promoter and the terminator of the cauliflower mosaic virus (CaMV) 35S promoter (Odell et al., (1985) “Identification of DNA sequences required for activity of the cauliflower mosaic virus 35S promoter”. Nature, 313: 810-812p), as well as other promoters.

Alteration in xylans biosynthesis can be obtained by increasing the expression of the ugd gene encoding the enzyme UDP-glucose dehydrogenase. This enzyme is considered a key enzyme as it regulates the carbon flow for the production of starch and sucrose, and it is also regulated by myo-inositol availability.

Thus, in an embodiment of the present invention, the ugd gene of UDP-glucose dehydrogenase from Eucalyptus grandis was cloned in order to overexpress this gene in tobacco and Eucalyptus spp. plants, and in the other plants of interest.

In another embodiment, the objective of cloning the uxs 1 gene responsible for the production of the enzyme UDP-D-glucuronate carboxylase from trees was to use to change UDP-D-xylose production in trees and plants. Therefore, it is possible to evaluate the impact of overexpressing this gene on cell wall formation, and the effect on the development of the plants.

The UDP-D-xylose biosynthesis, an important precursor in hemicellulose synthesis, is mediated by the enzyme UDP-D-glucuronate carboxylase (EC 4.1.1.35), that transforms UDP-D-glucuronate into UDP-D-xylose in an irreversible reaction (Bar-Peled et al, (2001) “Functional cloning and characterization of a UDP-glucuronic acid decarboxylase: The pathogenic fungus Cryptococcus neoformans elucidates UDP-xylose synthesis”. Proc. Natl. Acad. Sci., USA, 98(21): 12003-12008).

With the purpose of altering the metabolic regulation of hemicellulose biosynthesis, an object of the present invention was to clone the genes responsible for the production of the target enzymes, from trees and plants, in order to later affect the overexpression or repression of these genes in cell wall polysaccharides biosynthesis and in the development of tobacco plants (Nicotinana tabacum), and of trees, with focus on Eucalyptus spp., among other plants.

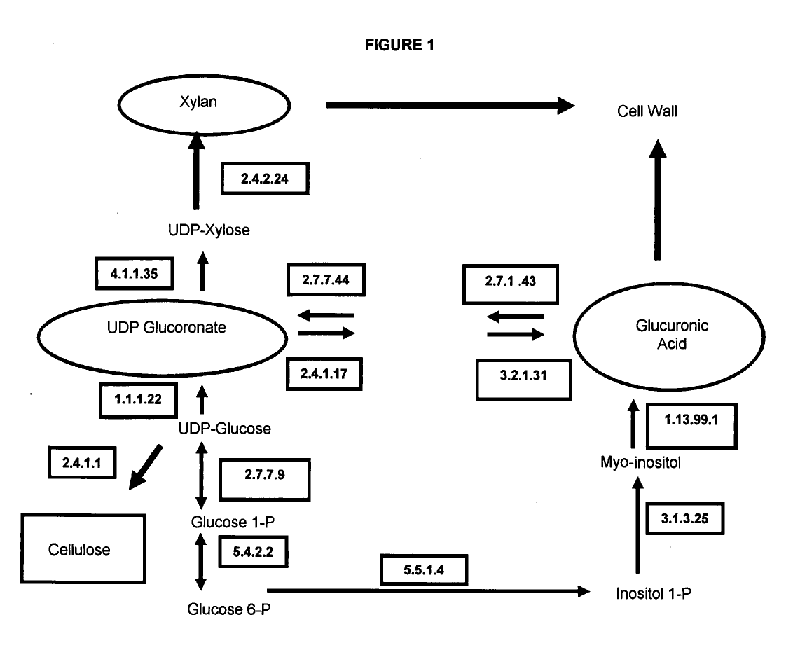

The method, according to the present invention, is further explained by means of FIG. 1 that schematically shows the metabolic pathway for the biosynthesis of hemicelluloses, cellulose and/or uronic acids, as well as the formation of xylans and glucuronic acids from the sugar Glucose 6-P and the pathway for the oxygenation of myo-inositol.

In yet another aspect, the invention refers to a method of modulating polypeptide levels in plants, the said polypeptides being involved in the biosynthesis of hemicelluloses, cellulose and/or uronic acids, characterized by the following stages a) introduction of one or more gene expression cassettes that comprises one or more genes encoding one or more enzymes selected from the group that contains: myo-inositol 1-phosphate synthase (EC: 5.5.1.4), myo-inositol monophosphatase (EC: 3.1.3.25), myo-inositol oxygenase (EC: 1.13.99.1), β-glucuronidase (EC: 3.2.1.31), glucuronokinase (EC: 2.7.1.43), glucuronosyltransferase (EC: 2.4.1.17), glucuronate-1-phosphate uridyltransferase (EC: 2.7.7.44), phosphoglucomutase (EC: 5.4.2.2), UDP-glucose pyrophosphorylase (EC: 2.7.7.9), UDP-glucose dehydrogenase (EC: 1.1.1.22), UDP-D-glucuronate carboxylase (EC: 4.1.1.35), 1,4-β-D-xylan synthase (EC: 2.4.2.24), and cellulose synthase (EC: 2.4.1.1) in the plant cell; b) regeneration of the plant cell; c) induction of the expression of the said polypeptides during a sufficient period to modulate the levels of the biosynthesis of hemicelluloses, cellulose and/or uronic acids level in the said plants.

### EXAMPLES

The following examples are merely illustrative to present in an exemplified manner certain specific embodiments of the invention. They should not, however, be considered as limiting to their wider extension, which are only delimited by the enclosed claims.

### Example 1

### Total RNA Isolation

In this embodiment, total RNA was obtained with the help of the protocol by SALZMAN, R. A, FUJITA, T., ZHU-SALZMAN, K., HASEGAWA, P. M., BRESSAN, R. A. (1999) (An Improved RNA Isolation Method for Plant Tissues Containing High Levels of Phenolic Compounds or Carbohydrates—Plant Molecular Biology Reporter 17: 11-17, 1999).

Total RNA was isolated from eucalyptus roots. Eucalyptus roots, were macerated in liquid nitrogen, transferred to the extraction buffer, followed by two to three extractions with chloroform/isoamilic alcohol in the proportion of 24:1 (v/v), and centrifugation for the separation of the aqueous and organic phases. The proteins and carbohydrates remaining in the aqueous phase were precipitated with NaCl and ethanol, at −20° C., for at least 3 hours. After the addition of phenol:chloroform:isoamilic alcohol (25:24:1) to the supernatant resulting from the previous precipitation, a centrifugation was conducted to separation of the aqueous phase from the organic phase. The second precipitation with NaCl and ethanol was performed at −20° C., also for at least 3 hours. The pellet containing total RNA was ressuspended in DEPC (diethyl pyrocarbonate) water, followed by precipitation with lithium chloride. The precipitate was washed in 80% ethanol, before quantification by spectrophotometry at a wavelength of 260 nm, and verification 1% agarose gel in 1×buffer TAE.

### Example 2

### Messenger RNA Extraction (mRNA)

The messenger RNA was isolated using the mRNA purification kit named “Dynabeads”, of the American company DYNAL Biotech, according to the manufacturer's standards.

### Example 3

### Specific Primers Synthesis

To the sequence of the sense primer of the superexpressed genes (UDP-glucose dehydrogenase, UDP-D-glucuronate carboxylase, UDP-glucose pyrophosphorylase) was associated the sequence CACC immediately before the 5′ region, that is, to the translational start codon.

To the sequence of the antisense primer of the myo-inositol oxygenase gene was associated the sequence CACC immediately after the 3′ region of the primer, that is to the translational stop codon.

### Example 4

### cDNA Synthesis

The cDNA synthesis, followed by amplification with specific primers corresponding to ORF's of these genes was accomplished, in a single step, using the Invitrogen kit called “SuperScript™ One-Step RT-PCR with Platinum Taq”, according to the manufacturer's standards.

### Example 5

### RT-PCR Reaction Product Cloning

Cloning of the product resulting from the amplification reaction with the specific primers that completely flank the ORF's was accomplished using the Invitrogen kit called “pENTR-Directional TOPO Cloning Kit”, according to the manufacturer's standards.

### Example 6

### LR Clonase Recombination Reaction

The recombination reaction, in this particular case, between the chimeric construction of the pENTR vector and the Gateway binary vector for plant transformation, was mediated by LR Clonase of the Invitrogen (Karimi, M. et al., above).

### Example 7

### Transformation Method

The method for obtaining genetically modified plants from eucalyptus established in the Physiologic Genetics Laboratory of the Genetics Department of the Superior School of Agriculture “Luiz de Queiroz” of the University of São Paulo (USP), according to Brazilian patent application PI0003908, uses the Agrobacterium tumefaciens-mediated plant transformation method for the introduction of the genes. The plant material used is the eucalyptus seed. The seeds are sterilized prior to sowing in a MS (Murashige and Skoog, 1962) medium, where they remain for 15 days, until the development of plantules. The cotyledons of these plantules are the explants to be inoculated with A. tumefaciens. Apart from the gene of interest, the constructions used have the NPTII gene of selective resistance to the antibiotic kanamycin. The bacterium is cultivated during 24 hours in liquid AB medium until it reaches an optic density close to 0.8 (OD_{660 nm}). The cotyledons extracted from the plantules are inoculated with the bacterial culture for 6 hours. After this period, the explants are transferred to a solid MS medium, for a culture period of 48 hours. The cotyledons are then transferred to a callus formation medium (MS with 5 μM TDZ and 5 μM ANA) containing 50 mg/L kanamycin and 400 mg/L cefotaxime. The calli that have developed are transferred to a shoot formation medium (MS with 5 μM TDZ and 2.5 μM ANA) with 25 mg/L kanamycin. Shoots that present a satisfactory development are elongated and rooted. The plants that develop from these shoots are then kept under controlled conditions of temperature, humidity, and light in a green house. The presence of the transgene is confirmed via the Southern Blot technique, using the genomic DNA of the transformants.

### Example 8

a) Determination of the transgene pattern expression was accomplished via Northern Blot. Total RNA was isolated from transformant plants, deriving form independent transformation events, according to example 5. The RNA samples were analyzed via Northern Blot for the determination of the pattern expression of each transgene in relation to independent transformation events.

b) Determination of the concentration level of xylans and glucuronic acid, after attainment and growth of the transformed plants.

1. GENE EXPRESSION CASSETTE, characterized by the fact that comprises one or more genes encoding one or more enzymes selected from the group that contains: myo-inositol 1-phosphate synthase (EC: 5.5.1.4), myo-inositol monophosphatase (EC: 3.1.3.25), myo-inositol oxygenase (EC: 1.13.99.1), β-glucuronidase (EC: 3.2.1.31), glucuronokinase (EC: 2.7.1.43), glucuronosyltransferase (EC: 2.4.1.17), glucuronate-1-phosphate uridyltransferase (EC: 2.7.7.44), phosphoglucomutase (EC: 5.4.2.2), UDP-glucose pyrophosphorylase (EC: 2.7.7.9), UDP-glucose dehydrogenase (EC: 1.1.1.22), UDP-D-glucuronate carboxylase (EC: 4.1.1.35), 1,4-β-D-xylan synthase (EC: 2.4.2.24), and cellulose synthase (EC: 2.4.1.1).

2. CASSETTE, according to claim 1, characterized by the fact that the said group contains: myo-inositol oxygenase (EC: 1.13.99.1), β-glucuronidase (EC: 3.2.1.31), glucuronokinase (EC: 2.7.1.43), glucuronate-1-phosphate uridyltransferase (EC: 2.7.7.44), UDP-glucose pyrophosphorylase (EC: 2.7.7.9), UDP-glucose dehydrogenase (EC: 1.1.1.22), and UDP-D-glucuronate carboxylase (EC: 4.1.1.35).

3. CASSETTE, according to claim 1, characterized by the fact of being cloned into the transformation binary vector and introduction into the bacterium Agrobacterium tumefaciens.

4. CASSETTE, according to claim 1, characterized by the fact that the said enzymes are involved in the biosynthesis of hemicelluloses, cellulose and/or uronic acids.

5. CASSETTE, according to claim 4, characterized by the fact that the hemicelluloses are xylans.

6. CASSETTE, according to claim 4, characterized by the fact that an uronic acid is glucuronic acid.

7. USE OF ONE OR MORE GENE EXPRESSION CASSETTES, characterized by the fact that it is for the overexpression or repression of the genes described in claim 1.

8. METHOD FOR GENETIC TRANSFORMATION IN PLANT CELLS, characterized by the fact of introducing one or more cassettes, according to one of claim 1, into the plant genome.

9. METHOD, according to claim 8, characterized by the fact that the cassette is introduced in the cell by electroporation, bioballistics, microinjection, macroinjection or via Agrobacterium tumefaciens.

10. METHOD, according to claim 9, characterized by the fact that the cassette is introduced into the cell by Agrobacterium tumefaciens.

11. METHOD, according to claim 8, characterized by the fact of changing the metabolic pathway for the biosynthesis of hemicelluloses, cellulose and/or uronic acids.

12. METHOD, according to claim 8, characterized by the fact that the said plant cell is a cell from any part of the plant, such as the root, stem, fruit, leaf, seed, or flower.

13. METHOD TO OBTAIN GENETICALLY MODIFIED PLANT, characterized by the fact that comprises the following stages:
a) genetic transformation of plant cells according to claim 8;
b) regeneration of stage (a) cells;
(c) expression of the DNA introduced into the cells of stage (b) in sufficient amount to substantially change the metabolic pathway for the biosynthesis of hemicelluloses and/or cellulose and/or uronic acids; and
d) obtention of the modified plant.

14. METHOD, according to claim 13, characterized by the fact that the said modified plant is a cell, an organ, a tissue, a seed, the entire plant, or its derived plants.

15. GENETICALLY MODIFIED PLANT, characterized by the fact of containing one or more expression cassettes according to claim 1.

16. GENETICALLY MODIFIED PLANT, characterized by the fact of being originated from the method according to claim 13.

17. PLANT, according to claim 15, characterized by the fact of being an angiosperm.

18. PLANT, according to claim 15, characterized by the fact of being a gymnosperm.

19. PLANT, according to claim 17, characterized by the fact of being Eucalyptus.

20. USE OF THE PLANT, according to claim 15, characterized by the fact of being used for obtaining wood and/or cellulose.

21. DERIVED PLANTS, characterized by the fact of being originated from the genetically modified plant, according to claim 15.

22. GENETICALLY MODIFIED SEED, characterized by the fact of comprising one or more expression cassettes according to claim 1.

23. GENETICALLY MODIFIED SEED, modified by introducing one or more cassettes into the genome, with the one or more expression cassettes comprised according to claim 1.

24. GENETICALLY MODIFIED SEED, obtained from the method of claim 13, characterized by the fact of presenting change in the biosynthesis of cellulose, hemicelluloses and/or uronic acids.

25. USE OF THE GENETICALLY MODIFIED SEED, according to claim 22, characterized by the fact of being used to generate plants.

26. WOOD, characterized by the fact of being obtained from the genetically modified plant according to claim 15.

27. USE OF THE WOOD, obtained from the genetically modified plant according to claim 15, characterized by the fact of being destined for civil engineering, shipbuilding, furniture and utensil manufacturing, and for the production of cellulose pulp and paper.

28. USE OF THE WOOD, according to claim 27, characterized by the fact of being for the production of cellulose pulp and paper.

29. CELLULOSE, characterized by the fact of being obtained from the wood according to claim 26.

30. USE OF THE CELLULOSE, obtained from the wood according to claim 26, characterized by the fact of being used for the manufacture of paper.

31. PAPER, characterized by the fact of being obtained from the wood or cellulose according to claim 26, respectively.

32. USE OF THE PAPER, obtained from the wood or cellulose according to claim 26, respectively, characterized by the fact of being for civil engineering, furniture industry, printing paper, packaging and absorbing paper.

33. USE OF THE PAPER, obtained from the wood or cellulose according to claim 26, characterized by the fact of being for printing paper, packaging and absorbing paper.

34. METHOD FOR MODULATING THE POLYPEPTIDES LEVEL IN PLANTS, the said polypeptides being involved in the biosynthesis of hemicelluloses, cellulose and/or uronic acids, characterized by the fact of comprising the following stages: a) introduction of one or more gene expression cassettes into the plant cell according to claim 1; b) regeneration of the plant cell; c) induction of the expression of the said polypeptides during a sufficient period to modulate the level of biosynthesis of hemicelluloses, cellulose and/or uronic acids in the said plants.


SEQUENCE LISTING (4 sequences)

SEQ ID NO: 1 - protein, 480 aa, Glycine max
MVKICCIGAGYVGGPTMAVIALKCPSIEVAVVDISKSRIAAWNSDQLPIYEPGLDGVVKQCRGKNLFFSTDVEKHVFEADIVFVSVNTPTKTQGLGAGKAADLTYWESAARMIADVSKSDKIVVEKSTVPVKTAEAIEKILTHNSKGIKFQILSNPEFLAEGTAIKDLFNPDRVLIGGRETPEGQKAIQTLKDVYAQWVPEERILTTNLWSAELSKLAANAFLAQRISSVNAMSALCEATGANVQQVSYSVGTDSRIGPKFLNASVGFGGSCFQKDILNLVYICECNGLPEVAEYWKQVIKINDYQKSRFVNRVVASMFNTVSNKKIAILGFAFKKDTGDTRETPAIDVCQGLLGDKANLSIYDPQVTEDQIQRDLSMNKFDWDHPIHLQPTSPTTVKKVSVVWDAYEATKDAHGLCILTEWDEFKTLDYQKIFDNMQKPAFVFDGRNIVDADKLREIGFIVYSIGKPLDPWLKDMPAVA

SEQ ID NO: 2 - protein, 346 aa, Pisum sativum
MAANSSNGDNQKTSKQPPLPSPLRFSKFFQSNMRILVTGGAGFIGSHLVDRLMQNEKNEVIVADNYFTGSKDNLKKWIGHPRFELIRHDVTEPLMIEVDQIYHLACPASPIFYKYNPVKTIKTNVIGTLNMLGLAKRVGARILLTSTSEVYGDPLEHPQPETYWGNVNPIGVRSCYDEGKRVAETLMFDYHRQHGIEIRVARIFNTYGPRMNIDDGRVVSNFIAQALRDESLTVQSPGTQTRSFCYVSDLVDGLIRLMGGSDTGPINLGNPGEFTMLELAETVKELINPNVEIKIVENTPDDPRQRKPDITKAQELLGWEPKVKLRDGLPLMEGDFRLRLGIEKNN

SEQ ID NO: 3 - protein, 282 aa, Sus scrofa
MKDPDPSQVYRPDMDPEAAKDKGSFRNYTSGPLLDRVFRTYKLMHTWQTVDFVRKKHAQFGGFSYKRMTVLEAVDMLDGLVDESDPDVDFPNSFHAFQTAEGIRKAHPDKDWFHLVGLLHDLGKVLVLAGEPQWAVVGDTFPVGCRPQASVVFCDSTFQDNPDLQDPVYSTELGMYQPHCGLENALMSWGHDEYMYQMMKFNKFSLPGEAFYIIRFHSFYPWHTGGDYRQLCNEQDLAMLPWVQEFNKFDLYTKGSDMPDVDELRPYYQGLIDKYCPGVLCW

SEQ ID NO: 4 - protein, 477 aa, Solanum tuberosum
MVTATTLSPADAEKLNNLKSAVAGLNQISENEKSGFINLVGRYLSGEAQHIDWSKIQTPTDEVVVPYDKLAPLSEDPAETKKLLDKLVVLKLNGGLGTTMGCTGPKSVIEVRNGLTFLDLIVKQIEALNAKFGCSVPLLLMNSFNTHDDTLKIVEKYANSNIDIHTFNQSQYPRLVTEDFAPLPCKGNSGKDGWYPPGHGDVFPSLMNSGKLDALLAKGKEYVFVANSDNLGAIVDLKILNHLILNKNEYCMEVTPKTLADVKGGTLISYEGKVQLLEIAQVPDEHVNEFKSIEKFKIFNTNNLWVNLSAIKRLVEADALKMEIIPNPKEVDGVKVLQLETAAGAAIKFFDRAIGANVPRSRFLPVKATSDLLLVQSDLYTLTDEGYVIRNPARSNPSNPSIELGPEFKKVANFLGRFKSIPSIIDLDSLKVTGDVWFGSGVTLKGKVTVAAKSGVKLEIPDGAVIANKDINGPEDI